(19) United States
(12) Patent Application Publication
(10) Pub. No.: US 2026/0357203 A1
(43) Pub. Date: Nov. 12, 2026

(54) BIO-PRINTED SKIN MODEL, RELATED THREE-DIMENSIONAL PRINTING PROCESS AND DIRECT PERFUSION DEVICE PROVIDED WITH SAID MODEL

(71) Applicant: BIO SYSTEM LAB S.R.L., Treviso (TV) (IT)

(72) Inventors: Lorenzo Maria VISENTIN, Treviso (TV) (IT); Federico MAGGIOTTO, Treviso (TV) (IT); Elisa CIMETTA, Padova (PD) (IT)

(21) Appl. No.: 19/490,678

(22) PCT Filed: Jun. 5, 2024
(86) PCT No.: PCT/IB2024/055478

§ 371 Date: Dec. 5, 2025

(30) Foreign Application Priority Data
Jun. 6, 2023 (IT) 102023000011436

(51) Int. Cl. C12N 5/071; B33Y 10/00; B33Y 80/00; C12M 1/00

(52) U.S. Cl. CPC C12N 5/0629; C12M 29/10; B33Y 10/00; B33Y 80/00; C12N 2502/1323; C12N 2502/28; C12N 2513/00; C12N 2533/30; C12N 2533/54

(57) ABSTRACT

The bio-printed skin model (1) comprises:—at least one dermal layer (2) comprising at least one polymer matrix and a mixture of fibroblasts and endothelial cells and configured to simulate a human dermal tissue;—at least one epidermal layer (3) comprising at least one mixture of keratinocytes and configured to simulate a human epidermal tissue; wherein the dermal layer (2) comprises at least one inner duct (4) formed by fibroblasts and endothelial cells, configured to simulate a vascular channel of human skin and provided with at least two end stretches communicating with the outside and connectable to a system for active perfusion of substances.

### TECHNICAL FIELD

The present invention relates to a bio-printed skin model, a related three-dimensional printing process and a direct perfusion device provided with said model.

### BACKGROUND ART

The skin is the largest organ in the human body, accounting for about 16% of the body weight, and plays a key role in protecting the body from the external environment. Its layered structure, composed of epidermis, dermis and subcutis, is essential for its functions as a protective, thermoregulation and homeostasis physical barrier.

In recent years, the possibility of artificially recreating skin models has seen considerable development as such models enable in vitro drug trial studies, potentially speeding up preclinical drug testing phases and entering the clinical setting with the implantation of patches in patients who have suffered severe tissue injury.

Among the wide range of recently developed techniques for the fabrication of tissue substitutes, 3D bioprinting is becoming increasingly popular in clinical and research settings. In fact, 3D bioprinting technology enables customized deposition of cells embedded in a biomaterial (bioink) according to pre-processed digital models depending on the final performance required.

First and foremost among the known types of skin models are two-dimensional skin models, which consist of a single two-dimensional layer of cells or of a few overlapping cell sheets (Suhail, S. et al., Biotechnology Journal vol. 14(2019 )). Two-dimensional models are the most stable and easiest to use and are employed for culturing or co-culturing keratinocytes and immune cells.

Another known type of skin model is three-dimensional skin models. These have a separate three-dimensional structuring and subdivision of the various layers of the skin (epidermis, dermis and subcutaneous layer). These structures allow the formation of tight cell-cell bonds and intracellular junctions allowing exchanges of molecules, gases and nutrients between them and maintaining the structural integrity of the skin and the functionality thereof. In addition, the formation of the stratum corneum reduces the rate of drug diffusion and its bioavailability, mimicking the barrier function of human skin (Polini, A. et al., Drug Discovery, vol. 9, 335-352 (2014)).

Still, another known type of skin model is represented by skin-on-a-chip models. On-chip models are micro-fluidic devices with micrometer-sized housing chambers for dynamic cell culture in order to mimic the physiology of a tissue or of an organ (Bhatia, S. N. & Ingber, D. E., Nature Biotechnology, vol. 32, 760-772 (2014)). The improved control of the cellular microenvironment and the ability to apply physical or chemical stimuli to the tissue inside help to recreate physiology more accurately than in a static and traditional 3D culture. The application of these stimuli leads to changes in cell behaviors, with improved cell differentiation, better cell-cell and cell-matrix interactions and cell morphologies. In addition, on-chip models involve the use of porous microchannel-dividing substrates, allowing the study of tissue barrier functions and simulating tissue-tissue interfaces (Kim, H. J. et al., Lab Chip, 12, 2165-2174 (2012)).

The skin models developed and available to date have limitations that do not make the data obtained from the tests sufficiently valid without the mandatory counter-proof by means of an in vivo test.

In general, one of the most challenging and limiting aspects of skin models of known type is the difficulty of making vascular canals that effectively simulate the vascular network present in human skin. Indeed, in human skin, the wall of vascular canals greatly affects the exchange of substances between tissues and blood, thus creating a barrier effect.

In detail, two-dimensional skin models have a structural simplicity, characterized by a single layer or meager layers of cells, which makes this model unsuccessful in recreating the three-dimensional structural complexity and the cell-cell and cell-matrix interactions that exist in a body or parts of a body at both physiological and biological levels, thus limiting the accuracy in being able to predict the complicated effects of a drug, resulting from the cellular metabolism of the aforementioned skin. Also absent in two-dimensional cell cultures is the barrier effect in the stratum corneum that is created in 3D structures and better mimics slowing the diffusion of molecules and active ingredients. It follows that two-dimensional skin models cannot be considered representative of the chemical, physical and biological dynamics involved within the same sample obtained from vivo or in vivo.

On the other hand, three-dimensional skin models of known type have major limitations related to various aspects, such as the lack of vasculature for nutrient supply, oxygen, waste removal or nutrient concentration gradient, leukocyte trafficking and transdermal penetration of drugs into the bloodstream, the weak barrier properties and the lack/shortage of skin adnexa (sweat glands, hair follicles) and, finally, the inability to offer precise control over spatial-temporal chemical gradients and over physical environmental factors (temperature, mechanical forces, gases), making the sampling complicated of luminal contents for the analysis of drug adsorption, distribution, elimination and toxicity (ADMET) and the collection of cellular components at specific locations for extended biological analysis.

As far as skin-on-a-chip models are concerned, in these the micro-scale of the devices and the small portion of tissue used (micro-fragments, single sheets or clusters of cells) make such a model deficient at the quantitative level, in commensuration with the real value of interactions that develop in vivo within the complete and complex three-dimensional structures characterizing a biological system.

It is clear, then, that the skin models of known type are susceptible to considerable improvement.

### DESCRIPTION OF THE INVENTION

The main aim of the present invention is to devise a bio-printed skin model, a related three-dimensional printing process and a direct perfusion device provided with said model which allow effectively simulating the human skin both structurally and functionally.

Another object of the present invention is to devise a bio-printed skin model, a related three-dimensional printing process and a direct perfusion device provided with said model which allow in vitro tests to be performed that are representative of real human skin behavior.

A further object of the present invention is to devise a bio-printed skin model, a related three-dimensional printing process and a direct perfusion device provided with said model which allow avoiding the need for additional in vivo tests.

Another object of the present invention is to devise a bio-printed skin model, a related three-dimensional printing process and a direct perfusion device provided with said model which allow the aforementioned drawbacks of the prior art to be overcome within the framework of a simple, rational, easy and effective to use as well as cost-effective solution.

The aforementioned objects are achieved by this bio-printed skin model having the characteristics of claim 1.

The aforementioned objects are further achieved by this three-dimensional printing process for the production of skin models having the characteristics of claim 9.

The aforementioned objects are further achieved by this direct perfusion device provided for bio-printed skin models having the characteristics of claim 15.

### BRIEF DESCRIPTION OF THE DRAWINGS

Other characteristics and advantages of the present invention will become more apparent from the description of a preferred, but not exclusive, embodiment of a bio-printed skin model, a related three-dimensional printing process and a direct perfusion device provided with said model, illustrated by way of an indicative, yet non-limiting example in the accompanying tables of drawings in which:

FIG. 1 is a top view schematic representation of the bio-printed skin model according to the invention;

FIG. 2 is a schematic perspective representation of the bio-printed skin model;

FIG. 3 is a perspective view of a perfusion support provided with bio-printed skin models;

FIG. 4 is an exploded view of a direct perfusion device according to the invention;

FIG. 5 is a perspective view of a tank of a direct perfusion device;

FIG. 6 is a schematic representation from above of a direct perfusion device;

FIGS. 7-9 represent characterization graphs of polymerizable materials according to the invention;

FIGS. 10-12 represent characterization graphs of the bio-printed skin model.

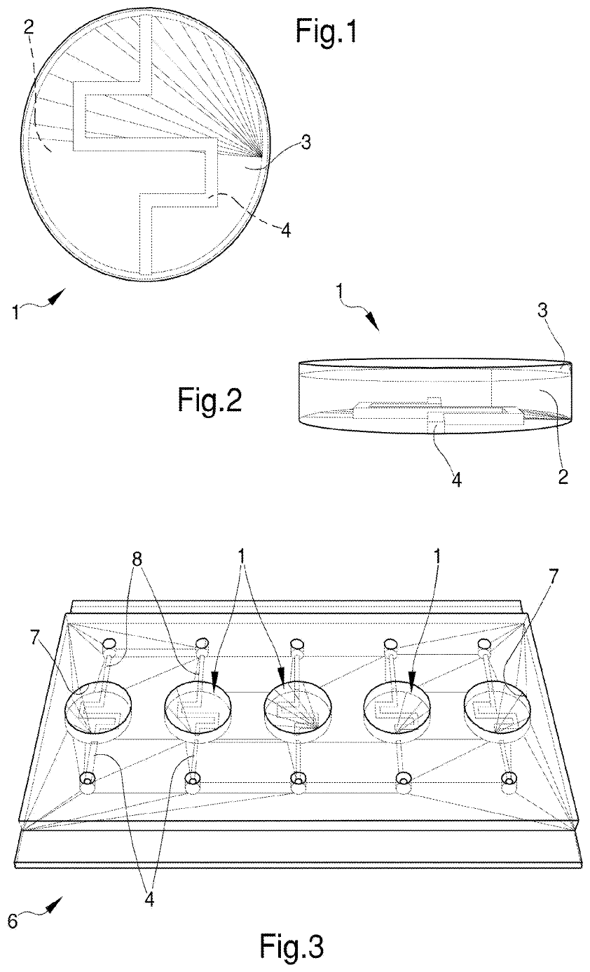

### EMBODIMENTS OF THE INVENTION

With particular reference to these figures, reference numeral 1 globally denotes a bio-printed skin model.

The skin model 1 according to the invention comprises:
at least one dermal layer 2 comprising at least a first polymer matrix and a mixture of fibroblasts and endothelial cells and configured to simulate a human dermal tissue;
at least one epidermal layer 3 comprising at least one mixture of keratinocytes and configured to simulate a human epidermal tissue;
   wherein the dermal layer 2 comprises at least one inner duct 4 formed by fibroblasts and endothelial cells, configured to simulate a vascular channel of human skin and provided with at least two end stretches communicating with the outside and connectable to a system for active perfusion of substances.

This model is made by means of a three-dimensional printing process, as will be better described later in the disclosure, thus ensuring the fabrication of a cellular construct in three dimensions, in a fast, accurate, standardized and low-cost manner, and allowing controlled deposition of the biomaterials used in the process. This model features the same layered structure of epidermis and dermis found in tissue in vivo, thus ensuring the formation of tight intracellular bonds and junctions so that molecules, gases and nutrients can be exchanged between them.

In detail, fibroblasts, endothelial cells and keratinocytes are live cells, and the inner duct 4 can be perfused with a working solution, which may consist of nutrients, oxygen or other substances intended to be transferred to the cells themselves for their sustenance. The working solution may also contain active substances, such as drugs or other molecules, in order to carry out bioavailability, bioequivalence tests and, in general, ADME (Absorption, Distribution, Metabolism, Elimination) tests.

These results are achievable due to the presence of the inner duct 4, which simulates an actual blood vessel found in human skin. The inner duct 4, in fact, is not only a channel formed in the dermal layer 2 but also has a coating wall formed by live cells. In addition, the use of fibroblasts in combination with the endothelial cells makes it possible to improve the biomechanical properties of the inner duct 4.

This skin model 1, therefore, is able to simulate the barrier effect which is normally present in blood vessels and that regulates the exchange of substances between blood and tissues by bringing oxygen, therefore, to the layers 2, 3, thus allowing the removal of waste substances or the formation of a nutrient concentration gradient, regulating leukocyte trafficking and transdermal penetration of drugs into the bloodstream.

In addition to live cells, the dermal layer 2 also comprises the polymer matrix that has a sustaining function for fibroblasts and endothelial cells.

Advantageously, the polymer matrix comprises photopolymerized methacrylate gelatin.

Methacrylate gelatin, also abbreviated as “GelMA”, is a gelatin-based hydrogel functionalized with methacrylate groups that cross-link in the presence of a photo-initiator. GelMA is a bioink widely used in bioprinting processes due to its biocompatibility, no-cytotoxicity and the presence of arginylglycylaspartic acid (RGD) for integrin binding and matrix metalloproteinase-sensitive groups for cell adhesion and migration (Bova, L. et al., Macromol Biosc (2022)).

In accordance with a first embodiment, the epidermal layer 3 in turn comprises a polymer matrix and the keratinocyte mixture.

In the following disclosure, the polymer matrix of the dermal layer 2 will be referred to as the “first polymer matrix” while the polymer matrix of the epidermal layer 3 will be referred to as the “second polymer matrix”.

The second polymer matrix also comprises photopolymerized methacrylate gelatin.

Still referring to the first embodiment, the dermal layer 2 and the epidermal layer 3 comprise GelMA at two different concentrations. This allows a stiffness gradient to be established between the two layers 2, 3, so as to mimic the viscoelastic properties of the tissue in vivo.

Conveniently, the first polymer matrix comprises methacrylate gelatin in saline buffer, in a concentration comprised between 5% and 10% m/V. Preferably, the first polymer matrix comprises methacrylate gelatin in saline buffer, in a concentration of 8% m/V.

The second polymer matrix, on the other hand, comprises methacrylate gelatin in saline buffer, in a concentration comprised between 12% and 18% m/V. Preferably, the second polymer matrix comprises methacrylate gelatin in saline buffer, in a concentration of 15% m/V.

As a result, the second polymer matrix is denser and imparts greater rigidity to the epidermal layer 3.

As shown above, the layers 2, 3 also have live cells dispersed in the relevant polymer matrices.

In accordance with the preferred embodiment, fibroblasts belong to the BJ cell line, i.e., neonatal preputial fibroblasts. It cannot, however, be ruled out that fibroblasts belong to a different cell line, e.g., to the HDF cell line, i.e., primary dermal fibroblasts. Endothelial cells are derived from the HUVEC cell line, i.e., endothelial cells of the human umbilical cord. Keratinocytes are derived from the HEK cell line, i.e., human epidermal keratinocytes.

Conveniently, the dermal layer 2 comprises the first polymer matrix and the mixture of fibroblasts and endothelial cells in a ratio comprised between 60:40 and 80:20. Preferably, in a ratio of 70:30.

In the dermal layer 2, moreover, fibroblasts are present in a cell density comprised between 1 million cells/mL and 2 million cells/mL and endothelial cells in a cell density comprised between 0.25 million cells/mL and 0.75 million cells/mL with respect to the first polymer matrix.

In the epidermal layer 3, on the other hand, keratinocytes are present in a cell density comprised between 5 million cells/mL and 10 million cells/mL.

The cell density and the ratio of cells to their respective polymer matrices were appropriately selected in order to obtain a good cell population while imparting sufficient rigidity to the relevant layers 2, 3.

In accordance with the embodiment shown in the figures, the dermal layer 2 has a thickness comprised between 0.7 mm and 1.3 mm.

The epidermal layer 3, on the other hand, has a thickness comprised between 0.1 mm and 0.5 mm.

The inner duct 4 comprises fibroblasts and endothelial cells in a ratio comprised between 15:85 and 35:65. Preferably, in a ratio of 30:70.

In accordance with a preferred embodiment, the inner duct 4 comprises a cross section having a characteristic dimension comprised between 0.3 mm and 0.8 mm. Specifically, if the inner duct 4 has a circular cross-section, the characteristic dimension is represented by the diameter of the cross-section, while if the inner duct 4 has a rectangular cross-section, the characteristic dimension is represented by the length of one of the sides of the cross-section.

In accordance with a second embodiment of this skin model, the epidermal layer 3 comprises the keratinocyte mixture alone. Again, keratinocytes are present in a cell density comprised between 5 million cells/mL and 10 million cells/mL.

According to a further aspect, the present invention also relates to a three-dimensional printing process for the production of skin models.

The process according to the invention first comprises a phase of supply of a three-dimensional bioprinter and of a three-dimensional digital model of vascularized skin.

Preferably, the three-dimensional bioprinter is of the type of an extrusion printer and is provided with at least three independent print heads mounted on a three-axis movement system. The bioprinter has a temperature control system for controlling the print bed and the print heads.

The three-dimensional digital model has been designed using digital drawing programs of known type.

The process also comprises a phase of supply of:
at least one bioink comprising a mixture of a polymerizable material and live cells selected from the list comprising: fibroblasts and endothelial cells; and
at least one sacrificial bioink.

The process then comprises a phase of three-dimensional printing of the bioinks according to the digital model to obtain at least a first layer made in the first bioink and at least one track made in the sacrificial bioink, developing within the first layer and provided with at least two end stretches communicating with the outside.

Thereafter, the process comprises a phase of polymerization of the polymerizable material to obtain at least one dermal layer 2 comprising the polymer matrix and the mixture of fibroblasts and endothelial cells and within which the track develops.

The process then involves a phase of distribution of at least one mixture of keratinocytes on top of the first layer to obtain at least one epidermal layer 3.

Finally, the process comprises the phases of:
removal of the track to obtain a through cavity;
coating of the through cavity with fibroblasts and endothelial cells to obtain at least one inner duct 4 developing in the dermal layer 2 and configured to simulate a vascular canal of human skin; and
cell growth carried out by means of direct perfusion of a culture medium through the inner duct 4 to obtain a skin model 1.

In accordance with a first embodiment of this process, the phase of distribution is in turn carried out by means of three-dimensional printing. Thus, the phase of supply involves the supply of a first bioink comprising a mixture of a first polymerizable material, fibroblasts and endothelial cells intended for making the dermal layer 2 and of a second bioink comprising a mixture of a second polymerizable material and keratinocytes intended for making the epidermal layer 3.

The sacrificial bioink, on the other hand, is intended to make a through cavity within the dermal layer 2.

The first and the second bioinks comprise methacrylate gelatin that has not yet been polymerized, that is, in the form of a hydrogel suitable for extrusion. Methacrylate gelatin can be synthesized using the protocol developed by Shirahama et al. (Sci Rep, 6, (2016)), as will be better described later in this disclosure.

Each of the first bioink and the second bioink also comprises a photo-initiating agent. In accordance with the preferred embodiment, the photo-initiating agent is lithium phenyl-2,4,6-trimethylbenzoylphosphinate.

Each of the first bioink and the second bioink also comprises cell lines in the quali-quantitative compositions described above.

The sacrificial bioink, on the other hand, comprises a thermo-gelling polymer. Specifically, the sacrificial bioink is poloxamer 407. Poloxamer 407 is a synthetic copolymer with a peculiar thermo-reversibility, that is, it is in the liquid state at temperatures below 20° C. and is in gel form at higher temperatures.

Therefore, the printing phase is advantageously carried out at a temperature comprised between 20° C. and 30° C.

The process then comprises the phase of three-dimensional printing of bioinks according to the digital model. In accordance with the first embodiment, this phase makes it possible to achieve:
at least a first layer made in the first bioink;
at least one track made in the sacrificial bioink, developing within the first layer and provided with at least two end stretches communicating with the outside; and
at least one second layer made in the second bioink and placed on top of the first layer.

Next, the process involves a polymerization phase of the polymerizable materials to obtain:
at least one dermal layer 2 comprising the first polymer matrix and the mixture of fibroblasts and endothelial cells and within which the track is developed; and
at least one epidermal layer 3 comprising the second polymer matrix and the keratinocyte mixture.

Specifically, the first polymerizable material generates the first polymer matrix and the second polymerizable material generates the second polymer matrix.

The first polymerizable material is a solution of methacrylate gelatin in saline buffer in a concentration comprised between 5% and 10% m/V. Preferably, in a concentration of 8% m/V.

The second polymerizable material is a solution of methacrylate gelatin in saline buffer in a concentration comprised between 12% and 18% m/V. Preferably, in a concentration of 15% m/V.

Polymerizable materials also comprise lithium phenyl-2,4,6-trimethylbenzoylphosphinate in a concentration of 0.1% m/V.

The polymerization phase is carried out by means of irradiation of the bioinks with electromagnetic radiation. Electromagnetic radiation is selected from the list comprising: UV radiation and IR radiation.

In this case, the polymerization phase is performed by exposure to UV light (405 nm) for a time comprised between 1 min and 2 min.

In detail, the polymerization of the first layer and of the second layer is carried out simultaneously.

The process then comprises a phase of removal of the track to obtain a through cavity.

The removal phase comprises a cooling step of the track at a temperature of less than 20° C. Below this temperature, in fact, the sacrificial bioink is in the liquid phase and can be easily removed.

The phase of removal also comprises a washing step of the through cavity by means of a washing liquid. For example, the washing liquid can be a buffer solution, such as e.g. a phosphate buffer.

The use of a sacrificial bioink allows the formation of the through cavity and, later, of the inner duct 4, without jeopardizing in any way the structural integrity of the through cavity itself and allowing perfusion to actively stimulate the rooting of the endothelial cells and of the fibroblasts to form the coating wall of the inner duct 4.

The process subsequently comprises a phase of coating the through cavity with fibroblasts and endothelial cells to obtain the inner duct 4 developing in the dermal layer 2 and configured to simulate a vascular canal of human skin.

Specifically, the coating phase comprises a step of injecting a cell culture comprising fibroblasts and endothelial cells into the through cavity and a step of incubating the cell culture into the through cavity.

The cell culture comprises fibroblasts and endothelial cells in a mutual ratio comprised between 15:85 and 35:65 and at a total density comprised between 7 million cells/mL and 13 million cells/mL.

The cell culture incubation step is performed by cyclically flipping the layers 2, 3. By doing so, the cell culture cyclically coats the through cavity so as to promote even cell deposition.

Finally, the process comprises a cell growth phase carried out by means of direct perfusion of a culture medium through the inner duct 4 to obtain the skin model 1. Specifically, the culture medium is composed of equal parts of the culture media of the single cell lines.

The growth phase is carried out by means of a direct perfusion device 5.

Direct perfusion devices allow tissue models to be perfused continuously with solutions containing nutrients.

The skin model is contained in a device designed to ensure continuous perfusion of nutrients through the vascular canal.

Advantageously, the printing phase is carried out on a perfusion support 6 for direct perfusion devices 5.

In other words, the skin model 1 is made directly on a direct perfusion device.

For this purpose, the process also comprises a phase of attaching at least the first polymerizable material to the perfusion support 6.

In detail, at least the first polymerizable material comprises nucleophilic functional groups, in this case represented by the amine groups of the lysine residues of gelatin, and the attaching phase comprises a step of functionalization of the perfusion support 6 with electrophilic functional groups, performed prior to the printing phase.

The perfusion support 6 is of the type of a microscope slide made of biocompatible material. The perfusion support 6 is provided with at least one housing seat 7 intended to receive the polymerizable materials and with at least one inlet duct 8 and at least one outlet duct 9 for a working fluid, connected to the housing seat 7 in a fluid-operated manner.

In accordance with the preferred embodiment, the perfusion support 6 is made of polydimethylsiloxane (PDMS), with a crosslinker-to-silicone ratio of 1:20, through a replica molding technique and molded on a glass microscope slide.

The functionalization step then involves activating the surface of the perfusion support 6 made of polydimethylsiloxane with electrophilic functional groups, specifically free carbonyl groups. The free carbonyl groups establish covalent bonds with the amine functional groups of the gelatin, thus ensuring adhesion of the biomaterial to the microscope slide perfusion support.

In accordance with a second embodiment, this process differs from what previously described in that the phase of distribution is carried out by deposition of a mixture of keratinocytes in a culture medium above the first layer.

In this case, the phase of distribution is carried out after the phase of polymerization of the polymerizable material.

Following then cell proliferation, the epidermal layer 3 acquires a degree of rigidity similar to that of the human epidermis and to that which would be obtained in accordance with the first embodiment, by means of the second polymer matrix.

According to a further aspect, this invention also relates to a skin model 1 obtainable by the process according to one or more of the previously described embodiments.

According to a further aspect, this invention also relates to a direct perfusion device 5 for bio-printed skin models.

The direct perfusion device 5 according to the invention comprises at least one perfusion support 6 provided with at least one housing seat 7 and with at least one skin model 1 according to one or more of the above-described embodiments, arranged in the housing seat 7, and with at least one inlet duct 8 and with at least one outlet duct 9 for a working fluid, connected to the inner duct 4 in a fluid-operated manner.

In detail, each of the inlet duct 8 and the outlet duct 9 is connected to an end stretch of the inner duct 4.

The skin model 1 is, therefore, directly bio-molded in the housing seat 7.

According to the above, the skin model 1 is attached to the housing seat 7 by means of covalent bonds.

The direct perfusion device 5 also comprises an adjustable compression unit 10 adapted to contain the perfusion support 6. The direct perfusion device 5 also comprises a tank 11 adapted to contain the culture medium and connected to the perfusion support 6 through the adjustable compression unit 10. The direct perfusion device 5 also comprises at least one pumping unit 12 positioned between the tank 11 and the perfusion support 6 for the movement of fluids.

The flow of cell medium through the vascular canal was established by connecting the skin model 1 to a perfusion circuit. The perfusion support 6, arranged within the adjustable compression unit 10 is connected to the pumping unit 12 and to the tank 11 through gas-permeable silicone tubing with an inner diameter of 0.51 mm.

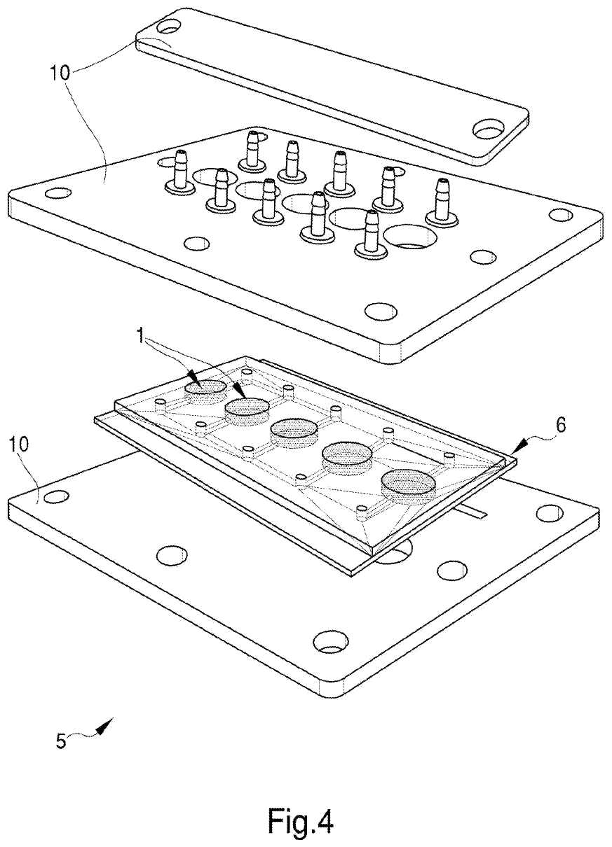

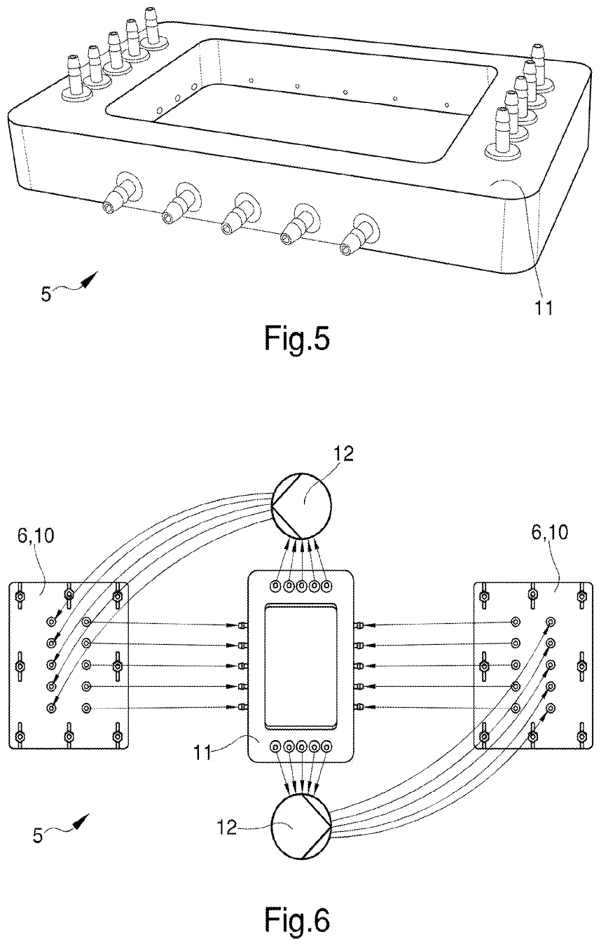

The perfusion support 6 is fitted within an adjustable compression unit (FIG. 4). This element has a dual purpose: to compress the PDMS to seal the skin model 1, thus preventing possible leakage of fluid, and to connect the perfusion support 6 to the tank 11 of the culture medium and to the pumping unit 12. Specifically, compression is carried out through butterfly screws, thereby moving the upper block of the adjustable compression unit 10 to the required height. The adjustable compression unit 10 is connected to the perfusion support 6 through adapters that fit snugly into the inlet and outlet ends of the culture medium present in the adjustable compression unit, thus limiting pressure drops in the stretch. The set of the adjustable compression unit 10 and the perfusion support 6 is connected to the pumping unit 12 and to the tank 11 of the culture medium through adapters into which the fluid movement tubing is directly fitted.

The culture medium is collected in the tank 11 and then recirculated to the skin models 1 in a closed loop. The tank 11 of the culture medium is capable of collecting up to 35 mL of medium, and the connection to the other elements of the loop is ensured by the presence of adapters that allow direct insertion of the fluid-carrying tubing. As shown in FIG. 6, the tank 11 is designed so that up to ten skin models, i.e., two perfusion supports 6, can be connected in parallel.

The fluid is taken from the tank 11 and is pumped through the skin models 1 using a peristaltic pump. The flow rate of the culture medium is changed during the experiment: the first two days of culture is kept at 15 μL/min, then increased to 30 μL/min for the following two days and finally raised to 45 μL/min until the tissue matures.

The direct perfusion device 5 allows the skin model to be maintained in active perfusion through the continuous supply of nutrients and oxygen via the inner duct 4, without the need to use porous membranes to recreate the contact membrane between fluid and cellular material, and without having to separate the various layers since the perfect physiology of the structures allows the transit of nutrients (via diffusion), within the cellular membranes. In addition, the wall of the inner duct 4, coated with endothelial cells, mimics the biophysical and biological characteristics of the vascular system in vivo.

Such a situation thus allows the testing of drugs and molecules, either topically, or by assessing the diffusion of the active ingredient within the flow or by mimicking the passages of the aforementioned ingredient between the various layers of tissue with the resulting physiological resistances. In addition, the physiologically relevant model size allows timely physiological and histological analyses to be performed in any area of the sample identified by the technician, overcoming a major limitation of current skin models.

### Experimental Part

As stated above, GelMA is chemically synthesized by functionalizing gelatin with methacrylic anhydride to enable photo cross-linking of the biomaterial. The key parameter to be monitored for GelMA synthesis is the degree of functionalization (DoF, or degree of substitution), which represents the percentage of lysine functionalized with methacrylate groups. The DoF was determined for each synthesized batch using proton magnetic resonance analysis (H-NMR).

### Gelma Synthesis

A 10% (w/v) solution of GelMA with a degree of functionalization (DoF) of 70% is prepared by initially dissolving a type A gelatin (~300 blooms from pig skin) in a 0.25 M carbonate-bicarbonate buffer (CB buffer) (sodium carbonate and anhydrous sodium carbonate in 1×PBS (phosphate buffer solution) for 20 min at 40° C. and constant stirring (800 rpm). The pH is adjusted to 9.2-9.4 by adding HCl (hydrochloric acid, 37%). When the solution is clear, 50 μL of methacrylic anhydride (94%) per gram of gelatin is added; the methacrylation reaction is maintained for 2 h at 40° C. and constant stirring (800 rpm). The reaction is then stopped by the addition of HCl, bringing the pH to a physiological value of 7.4. GelMA is then collected, centrifuged (3500 rpm, 5 min) and poured into dialysis membranes (14 kDa MWCO). Dialysis lasts for 5 days, keeping the membranes fully soaked in ultrapure water (milli-Q) at 40° C. with gentle and constant agitation (100-150 rpm); the dialysis water is replaced at least three times a day. GelMA is filtered through a 0.22-μm filter and loaded into a falcon with 0.22-μm filter cap before being lyophilized for 7 days. The lyophilized product can be stored at −20 °C until use, keeping it isolated from moisture.

### Gelma Characterization

Proton nuclear magnetic resonance (H-NMR). H-NMR spectra were recorded on a Bruker Avance spectrometer operating at 300.1 MHz with water suppression by means of pre-saturation. Solutions for analysis were prepared by dissolving ~20 mg of lyophilized GelMA in 0.50 mL of D 20(Spectra 2000 ) and stirring under gentle heating until the solid was completely dissolved.

Degree of functionalization (DoF). The DoF was determined by comparing the peak integrals related to the CH₂ protons of the lysine and hydroxylysine residues (at ≈2.90 ppm) in the methacrylate samples and in Bloom 300 pure gelatin (I_{GelMA} and I_{B300,} respectively), by using phenylalanine signals (6.50-7.40 ppm) as a reference for normalization. The presence of the methacrylate groups was confirmed by detecting the peaks of methylidenic protons (═CH₂) at ≈5.35 and 5.60 ppm.

The degree of functionalization is calculated by the following equation:

DoF=(1−(I_{GelMA}/I_{B300}))×100

### Preparation of polymerizable materials

Lithium phenyl-2,4,6-trimethylbenzoylphosphinate (LAP, 0.1% m/v) is dissolved in warm 1×PBS (40° C.) for 20 min with constant stirring (800 rpm), then the solution is filtered (0.22 μm filters) into a falcon containing lyophilized and weighed GelMA. The mixture is kept at 37° C. and stirred intermittently until GelMA is completely dissolved. Before use, the ink is centrifuged to remove air bubbles (3500 rpm, 5 min). The sacrificial bioink is composed of a solution of Pluronic F-127 (powder) in cold 1×PBS (4° C.). Intermittent mixing and stirring result in a clear solution.

### Characterization of Polymerizable Materials

Swelling capacity (SR). After photopolymerization, the samples were placed in petri dishes with buffer solution and incubated at 37° C. for 24 h to reach swelling equilibrium. The samples were then weighed to find their swollen masses, air-dried for 3 days and weighed again to measure the dried masses. The percent swelling ratio (SR) was evaluated with the following equation.

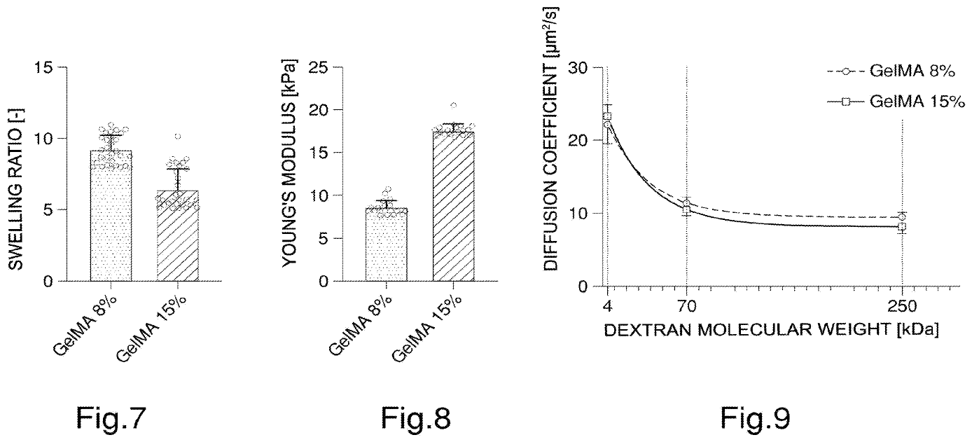

SR=((ms−md)/md)x·100%

where ms and md are the masses of the swollen and dried samples, respectively (FIG. 7). The results shown in FIG. 7 highlight that the higher weight content of GelMA in the hydrogel leads to lower SR values. In fact, the SR value for the first polymer matrix (GelMa 8%) is 9.19±1.01, while for the second polymer matrix (GelMa 15%) is 6.45±1.42 (with p value<0.001). This result is in line with expectations, as a higher weight content of GelMA results in less solvent in the hydrogel and, consequently, less weight related thereto.

Young's modulus. Young's modulus was derived from indentation data obtained with an atomic force microscope (Park Instruments XE-Bio AFM (South Korea)) provided with an inverted microscope (Nikon Eclipse Ti). Force-displacement curves were obtained using PPP-CONTSCR-10 pyramidal tips mounted on Si₃N₄ cantilevers with a nominal elastic constant of 0.2 N m⁻¹ (NanoSensors, Neuchatel, Switzerland). The elastic constants of cantilevers were calibrated by the manufacturer before use. Before each test, AFM photodetector sensitivity (optical lever sensitivity) was calculated by measuring the slope of the force-distance curve acquired on a silicon standard. All experiments were performed at room temperature, in a fluid environment (DPBS). Indentation curves were acquired by approaching the sample surface at a rate of 3 μm s⁻¹ and producing an indentation with a depth of 3 μm. At least six force curves were recorded at different locations for a minimum of three samples per condition. Young's modulus was calculated by applying a Hertz model fit to each individual force-distance curve, assuming a Poisson's ratio of 0.5. The evaluation of Young's modulus allows the mechanical properties of the polymer matrices to be determined and the similarities/differences thereof to real human tissues to be assessed. The results shown in FIG. 8 show a significant difference in the value of Young's modulus between the two materials (with p value<0.001): for the first polymerizable material (GelMa 8%) the value is 8.65±0.81 kPa, while for the second polymerizable material (GelMa 15%) it is 17.66±0.77 kPa. This result allows confirming the difference in stiffness between the two layers in the model, mimicking the physiology of skin in vivo.

Diffusion coefficient (D). The diffusion coefficient was obtained by fluorescence recovery after photobleaching (FRAP) assay. The assays were performed with dextran of different molecular weights (4 kDa, 70 kDa, 250 kDa) to measure the diffusion coefficient for a wider range of molecules. 100 μL of the first polymerizable material (GelMa 8%) and of the second polymerizable material (GelMa 15%) were cast and polymerized, incubated overnight in 4-kDa, 70-kDa, and 250-kDa dextran, and observed with a confocal microscope (ZEISS LSM 800). Images were acquired every 0.8 s for 90 s at 63× magnification after 20 cycles of bleaching (100% laser intensity) focused on a 20-μm radius region of interest. Six samples per hydrogel were processed and measurements were taken at five different locations for each sample; finally, a one-way variance analysis test was performed on the data obtained.

The diffusion coefficient was calculated by analyzing the average intensity data for the region of interest using the instantaneous bleaching correlation:

D=2r²/τ_{1/2}

where r is the radius of the region of interest and τ1/2 is the half-intensity recovery time (FIG. 9). The results in FIG. 9 confirm that the diffusion coefficient decreases with increasing molecular weight of the dextran by following a logarithmic trend, although without showing significant differences in the behavior between the two hydrogels. This allows stating that a high concentration of GelMA, as in the case of the second polymer matrix, does not jeopardize the mechanical properties of the material, thus ensuring an optimal value of the diffusion coefficient.

Printability analysis. Printability was evaluated following the printability parameter (Pr) introduced by Ouyang et al. (Biofabrication, 8, (2016)). The calculation was based on the equation that determines the circularity of a closed area:

C=4πA/L²

where L is the perimeter and A is the area, giving C=1 for a perfect circle; a square has maximum circularity at π/4. Therefore, the parameter Pr for square shapes can be calculated as in the equation:

Pr=π/4×1/C=L²/16A

According to this equation, Pr=1 when the area enclosed by the printed filaments is a perfect square. For hydrogels, a print with ideal viscosity will give a Pr=1, while Pr>1 will be associated with an overly gelled hydrogel with granular filaments and Pr<1 with a poorly gelled hydrogel that partially liquefies when printed. Grids with square closed spaces (n=6) were printed with each hydrogel and imaged by light microscopy. The images were then analyzed with ImageJ (Fiji) to extract the perimeter and inner area of the closed spaces and calculate Pr.

Bioinks were printed and tested under different conditions to optimize the printing parameters and multi-material printing protocol to reproduce the desired model. Gelatin-based hydrogels are highly temperature-dependent and identifying the optimal temperature value during the printing phase is critical to achieve a smooth, well-defined filament. To this end, different values were set in the temperature-controlled print head and monitored the aforementioned printability parameter. Bioinks were loaded into the extrusion syringes and mounted in the temperature-controlled print heads of an extrusion bioprinter. To test shape accuracy, hydrogels were printed in single-layer grids with square gaps between the filaments; calculation of the perimeter and of the area of the gaps provides the printability parameter, Pr. These tests allowed identifying the ideal printing temperatures for the different bioinks, nominally 23° C.±0.5° C. for the first bioink, 25° C.±0.5° C. for the second bioink and 25° C.±0.5° C. for the sacrificial bioink. Printing pressures relative to ideal temperatures are 50±10 kPa for the first bioink, 70±10 kPa for the second bioink and 90+5 kPa for the sacrificial bioink, using 25G conical nozzles for the first and second bioinks and a 27G conical nozzle for the sacrificial bioink. Under these conditions, the Pr values are 0.93±0.01 for the first bioink, 0.94±0.02 for the second bioink, and 0.96±0.03 for the sacrificial bioink.

The print bed was set at 22° C. throughout the process to promote the formation of the physical GelMA gel.

### Preparation of Cell Lines

BJ cells (preputial neonatal fibroblasts) were cultured in Eagle's Minimum Essential Medium (EMEM) supplemented with 10% FBS (Fetal Bovine Serum), 1% MEM (Minimum Essential Medium) and 1% P/S (Penicillin/Streptomycin), renewing the medium every 3 days. HUVEC (Human Umbelical Vein Endothelial Cells) cells were cultured in EGM-2 (Endothelial Cell Basal Medium 2) supplemented with growth factors (SupplementPack) and used until the 10th passage, by renewing the medium every 3 days.

HEK (human epidermal keratinocytes) cells were cultured in KBM (Keratinocyte Basal Medium) supplemented with growth factors (1%) and 1% P/S, renewing the medium every 3 days.

### Preparation of the First Bioink and the Second Bioink

The cell lines were then added to the polymerizable materials in a 1:25 ratio of cells suspended in medium to GelMA so as not to alter the properties of the hydrogel. The cell densities used for the dermis were 1.5 million cells/mL of first polymerizable material for fibroblasts and 0.5 million cells/mL of first polymerizable material for endothelial cells. The cell density used for the epidermis is 7 million cells/mL of second polymerizable material for keratinocytes.

### Functionalization of the Printing Medium

Functionalization involves activation of the surface by plasma treatment and, then, vapors of (3-aminopropyl)triethoxysilane (APTES) are bonded to the activated surface (2 h, vacuum gas phase). APTES is an amino-silane mainly used as a dispersion agent; it can attach an amine group to functional silane for bio-conjugation. At this point, 0.5% glutaraldehyde is added to bind to the amine groups (1 h, liquid phase). The free carbonyl group of the glutaraldehyde is then involved in forming the bond with the amine functional groups of the gelatin, thus ensuring adhesion of the biomaterial to the glass microscope slide.

### 3D Bioprinting

The bioinks are transferred into extrusion syringes and mounted on the bioprinter. The first bioink and the second bioink were mounted on temperature-controlled print heads with 25 G conical nozzles, while the sacrificial bioink did not require temperature control and was printed through a 27 G conical nozzle. The printing speed was set at 5 mm s⁻¹. After printing, the first layer and the second layer (when provided) were exposed to UV light (LED, 405 nm) for 90s to cross-link the structure.

### Preparation of the Inner Duct

Removal of the sacrificial bioink. Following printing, the layers were refrigerated at 4° C. for 5 min to liquefy the sacrificial bioink, which is then washed out by first injecting cold 1×PBS and then the culture medium, thus leaving the through channel.

Endothelialization of the inner duct. Inner duct endothelialization was performed following the post-seeding method after the printing process. Once the sacrificial bioink was washed from the through channel, HUVEC and BJ were injected at a total density of 10 million cells/mL medium in a ratio of 70:30%, respectively. The structure was then kept at 37° C. for 4 hours, turning it upside down every 30 minutes, before gently washing the unattached cells with warm medium. Finally, the structure was connected to the perfusion circuit and incubated at 37° C. and 5% CO₂.

### Skin Model Characterization

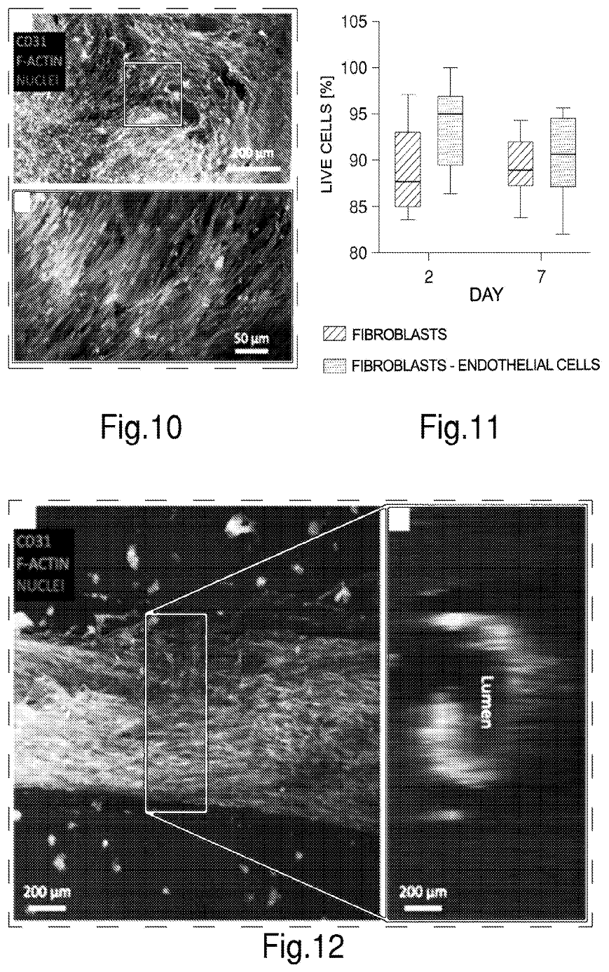

Cell viability assays. After cutting the model into four and performing three washing cycles in 1×PBS (5 minutes each), the samples were soaked in the staining solution for 45 minutes and incubated at 37° C. Subsequently, the samples were washed with 1×PBS (three washing cycles, 5 minutes each) before collecting images under a fluorescence microscope. Specifically, an image of the dermal layer at day 14 of perfusion is shown in FIG. 10.

FIG. 11 shows the graph of cell viability assessment at day 2 and day 7 (with p value<0.001).

FIG. 12 shows an image of the inner duct wall: (i) portion of the vascular canal at day 14 of perfusion, (ii) complete formation of the vascular lumen.

Fixation and staining. Vascularized structures were fixed and stained before being analyzed by confocal microscopy. The constructs were first washed with 1×PBS (3 cycles, 5 minutes each) and fixed in paraformaldehyde (PFA, 4% v/v) at 4° C. for 4 hours. After further washing with 1×PBS (3 cycles, 5 min each), they were permeabilized with PBS-T (Triton X-100 0.1% in 1×PBS) for 30 min. F-actin (1: 250, 45 min) and DAPI (1: 500, 15 min) staining were preceded by a rinse step with 1×PBS (3 cycles, 5 min each). Confocal microscopy was performed on a confocal microscope (ZEISS LSM 800), using spectral lasers at 561 nm and 455 nm.

Immunostaining. First, samples were washed with 1×PBS and fixed in 4% PFA for 4 hours at 4° C. After washes with PBS-T (Triton X-100 0.1% in 1×PBS), the samples were soaked overnight in a blocking solution consisting of 5% BSA (bovine serum albumin) in PBS-T. Diluted primary antibodies were then added into the blocking solution and the structures were incubated for 2 days. The samples were washed with PBS-T and the secondary antibodies diluted in the blocking solution were added and left to incubate overnight.

It has in practice been ascertained that the described invention achieves the intended objects, and in particular the fact is emphasized that the bio-printed skin model, the related three-dimensional printing process and the direct perfusion device provided with said model according to the present invention allow for the effective simulation of human skin both structurally and functionally.

In addition, the bio-printed skin model, the related three-dimensional printing process and the direct perfusion device allow in vitro tests to be performed that are representative of real human skin behavior.

Finally, this bio-printed skin model, the related three-dimensional printing process and the direct perfusion device make it possible to drastically reduce and thus largely replace the performance of additional in vivo tests.

1) Bio-printed skin model (1), characterized by the fact that it comprises:
at least one dermal layer (2) comprising at least one polymer matrix and a mixture of fibroblasts and endothelial cells and configured to simulate a human dermal tissue;
at least one epidermal layer (3) comprising at least one mixture of keratinocytes and configured to simulate a human epidermal tissue;
wherein said dermal layer (2) comprises at least one inner duct (4) formed by fibroblasts and endothelial cells, configured to simulate a vascular channel of human skin and provided with at least two end stretches communicating with the outside and connectable to a system for active perfusion of substances.

2) Skin model (1) according to claim 1, characterized by the fact that said polymer matrix comprises photopolymerized methacrylate gelatin.

3) Skin model (1) according to claim 1, characterized by the fact that said dermal layer (2) comprises said polymer matrix and said mixture of fibroblasts and endothelial cells in a ratio comprised between 60:40 and 80:20.

4) Skin model (1) according to claim 1, characterized by the fact that said dermal layer (2) comprises fibroblasts in a cell density comprised between 1 million cells/mL and 2 million cells/mL and endothelial cells in a density comprised between 0.25 million cells/mL and 0.75 million cells/mL with respect to said polymer matrix.

5) Skin model (1) according to claim 1, characterized by the fact that said polymer matrix comprises methacrylate gelatin in saline buffer, in a concentration comprised between 5% and 10% m/V.

6) Skin model (1) according to claim 1, characterized by the fact that said dermal layer (2) has a thickness comprised between 0.7 mm and 1.3 mm.

7) Skin model (1) according to claim 1, characterized by the fact that said inner duct (4) comprises fibroblasts and endothelial cells in a ratio comprised between 15:85 and 35:65.

8) Skin model (1) according to claim 1, characterized by the fact that said inner duct (4) comprises a cross section having a characteristic dimension comprised between 0.3 mm and 0.8 mm.

9) Three-dimensional printing process for the production of skin models, characterized by the fact that it comprises the following phases:
supply of a three-dimensional bioprinter;
supply of a three-dimensional digital model of vascularized skin;
supply of at least one bioink comprising a mixture of a polymerizable material and live cells selected from the list comprising: fibroblasts and endothelial cells, and of at least one sacrificial bioink;
three-dimensional printing of said bioinks according to said digital model to obtain at least a first layer in said bioink and at least one track in said sacrificial bioink, developing within said first layer and provided with at least two end stretches communicating with the outside;
polymerization of said polymerizable material to obtain at least one dermal layer (2) comprising at least one polymer matrix and one mixture of fibroblasts and endothelial cells and within which said track develops;
distribution of at least one mixture of keratinocytes on top of said first layer to obtain at least one epidermal layer (3);
removal of said track to obtain a through cavity;
coating of said through cavity with fibroblasts and endothelial cells to obtain at least one inner duct (4) developing in said dermal layer (2) and configured to simulate a vascular canal of human skin;
cell growth carried out by means of direct perfusion of a culture medium through said inner duct (4) to obtain a skin model (1).

10) Process according to claim 9, characterized by the fact that said printing phase is carried out at a temperature comprised between 20° C. and 30° C.

11) Process according to claim 9, characterized by the fact that said sacrificial bioink comprises a thermo-gelling polymer, said removal phase comprising a cooling step of said track at a temperature of less than 20° C., wherein said sacrificial bioink is in a liquid phase, and a washing step of said through cavity by means of a washing liquid.

12) Process according to claim 9, characterized by the fact that said coating phase comprises a step of injecting a cell culture comprising fibroblasts and endothelial cells into said through cavity and a step of incubating said cell culture into said through cavity.

13) Process according to claim 9, characterized by the fact that said growth phase is carried out by means of a direct perfusion device (5).

14) Process according to claim 9, characterized by the fact that said printing phase is carried out on a perfusion support (6) for direct perfusion devices (5) and by the fact that said process comprises a phase of attaching at least said first polymerizable material to said perfusion support (6).

15) Skin model (1) obtainable through the process according to claim 9.

16) Direct perfusion device (5) for bio-printed skin models, characterized by the fact that it comprises at least one perfusion support (6) provided with at least one housing seat (7) and with at least one skin model (1) according to claim 1 arranged in said housing seat (7), and with at least one inlet duct (8) and at least one outlet duct (9) for a working fluid, connected to said inner duct (4) in a fluid-operated manner.
